(19) United States
(12) Patent Application Publication
(10) Pub. No.: US 2026/0055378 A1
(43) Pub. Date: Feb. 26, 2026

(54) POWDER FORMULATIONS AND USE THEREOF IN MEDICAL AND/OR SURGICAL PROCEDURES

(71) Applicant: Marizyme, Inc., Jupiter, FL (US)

(72) Inventor: Cathy Pachuk, Jupiter, FL (US)

(21) Appl. No.: 18/855,944

(22) PCT Filed: Apr. 14, 2023
(86) PCT No.: PCT/US2023/018648

§ 371 Date: Oct. 10, 2024

Related U.S. Application Data
(60) Provisional application No. 63/331,777, filed on Apr. 15, 2022.

(51) Int. Cl. C12N 5/00

(52) U.S. Cl. CPC C12N 5/526

(57) ABSTRACT

A powder formulation for preserving tissue and organ function, and more particularly to shelf stable powder formulations that can be reconstituted at point of use, including during a fat grafting process, coronary artery bypass graft surgery, and peripheral bypass surgery, or other grafting/transplant surgery prior to or during the medical procedure to preserve the tissues/organs used in these procedures. The powder formulation includes a reducing agent; an antioxidant; a sugar; a nitric oxide substrate; a buffering agent; and physically acceptable salt. Also provided are a method of preparing an organ or tissue preservation solution by reconstituting the powder formulation, and a method of preserving tissue or an organ using same.

### FIELD OF THE INVENTION

The present invention is directed to powder formulations for preserving tissue and organ function, and more particularly to shelf stable powder formulations that can be reconstituted at point of use including during a fat grafting process, coronary artery bypass graft surgery, peripheral bypass surgery, and the like, prior to or during the medical procedure to preserve the tissues/organs used in these procedures.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions, or is known to be relevant to an attempt to solve any problem with which this specification is concerned.

The teachings of all the references cited herein are incorporated in their entirety by reference.

U.S. Pat. No. 7,981,596 discloses tissue and organ preservation solutions, generally called GALA™, and named after three primary components: glutathione, ascorbic acid and L-arginine. GALA™ solutions also include a balanced salt solution. These solutions are especially useful for preserving vascular conduits such as arteries and veins. Vascular conduits are used as grafts for a variety of bypass surgical procedures including but not limited to peripheral vascular bypass surgery and coronary artery bypass grafting (CABG) surgery.

Endothelial dysfunction is the primary determinant in the interrelated pathogenesis leading to vascular conduit failure. Graft failure is preceded by graft thrombosis, intimal hyperplasia and accelerated graft atherosclerosis, all of which are predicated upon previous functional and/structural impairments of the vascular conduit endothelium. It has been recognized that the choice of storage solution for intra-operatively harvested saphenous vein segments has a significant impact on endothelial structure and function and therefore graft patency. The inability of these solutions to adequately preserve the vascular conduit has been demonstrated by a number of investigators (Thatte et al., “Multi-photon microscopic evaluation of saphenous vein endothelium and its preservation with a new solution, GALA.” Ann Thorac Surg. 2003; 75(4):1145-1152; and Hussaini et al., “Evaluation of endoscopic vein extraction on structural and functional viability of saphenous vein endothelium.” J Cardiothoracic Surg 2011; 6:82-90). A 2011 ex vivo study by Wilbring et al. (Wilbring et al., “Even short-time storage in physiological saline solution impairs endothelial vascular function of saphenous vein grafts.” Eur J Cardiothoracic Surg. 2011; 40(4):811-815) demonstrated that vascular function was completely abolished after conduits harvested from patients were stored in a buffered saline solution, commonly used for vascular conduit storage. The data revealed that endothelial cell function was also significantly reduced yet these dysfunctional conduits were successfully used as grafts for bypass surgery in this study.

The effect of GALA™ preservation solution on human saphenous vein segments has also been evaluated in ex vivo studies. While solutions in clinical use today led to a profound decline in endothelial cell viability of vascular conduits (saphenous vein), GALA™ maintained endothelial function and structural viability for at least up to 24 hours (Thatte 2003, Hussaini 2011, and Thatte S. “Evaluation of endoscopic vein extraction on structural and functional viability of saphenous vein endothelium.” J Cardiothoracic Surg 2011; 6:82-90). The ex vivo data demonstrated that better preservation of vascular conduits could be afforded through the use of GALA™ as a vascular conduit preservation solution. However, the GALA™ solution has a limited shelf life due to instability of the GALA™ solution.

Fat grafting methods are performed in just over nineteen million US plastic surgery and related procedures (medical reconstructive/aesthetic) annually. Retention of fat grafts has a short term outlook, typically less than six months, and is a key area of focus for improved procedures and treatments in the plastics space. In an exemplary fat grafting process, homologous adipose tissue is removed by liposuction and is stored/processed outside the body for up to several hours prior to grafting. It has been well known in the art that there is a problem with graft retention, and it has been presumed to be caused by adipose tissue damage during liposuction and/or storage. The inventors of this application have found that ischemic injury specifically (which occurs during liposuction and subsequent tissue processing) and the use of tumescent solutions that are not fully biocompatible play an important role in tissue damage. Thus, there is a need to produce improved organ and tissue preservation solutions, preferably in the form of a tumescent fluid/solution, to be applicable to the preservation of fat grafts during the fat grafting process, including a preservation composition that has improved stability and shelf-life and is space-saving (i.e., powder or concentrated formulation) that can be reconstituted at point of use.

As described in U.S. Pat. No. 11,291,201, it has been shown that stability of the GALA™ solutions can be improved by separating the formulation into a first Solution A including a balanced solution and having a pH of at least 7, generally 7.4-8, and a Solution B having a pH of less than 7, preferably about 5, 4, or 3. Solution B includes water, an antioxidant such as ascorbic acid, a reducing agent such as L-glutathione, a nitric oxide substrate such as L-arginine and a sugar such as D-glucose. Solution B preferably has an oxygen content at zero parts per million. In other stable embodiments, the sugar can be added to Solution A instead of Solution B. However, the stable formulation described in U.S. Pat. No. 11,291,201 requires the manufacturing of two separate formulations that are combined at point of use, and a two-solution-containing kit with an aseptically manufactured Solution A bottle and Solution B vial containing the two separate solutions, respectively. Manufacture of the two-solution-containing kit requires two separate and costly manufacturing runs that comply with the US Food and Drug Administration's Good Manufacturing Practice regulations—one for Solution A and one for Solution B. Solutions A and B are manufactured under aseptic processing conditions. Solution B cannot be terminally sterilized because any form of terminal sterilization would destroy the organic components. The Sterility Assurance Level (SAL) of aseptically processed products is 10⁻³. Some medical indications, however, require a product with a lower SAL, such as 10⁻⁶, which cannot be reached with aseptic manufacturing processes. Furthermore, the manufacture of a product that includes two different solutions requires a separate cGMP manufacturing run for each solution on the product, thereby doubling the cost of the product.

Additionally, very large volumes of tumescent fluid are required during fat grafting procedures, oftentimes liters of solution, and it is impractical to have Solution A and Solution B manufactured as containers containing large volumes of solution. Interviews with plastic surgeons indicated the need for tumescent solution provided as a single bag solution, and at the same time related the impracticality of having additional bags containing a novel solution in operating room (OR) suites in which bags of saline and lactated ringer are already present.

Thus, there is a need for an organ or tissue preservation formulation that has high effectiveness and stability, has space-saving features, and can be prepared with reduced manufacturing and processing costs.

### SUMMARY

The present invention is directed to a powder formulation which, when reconstituted, can prevent ischemic injury during a fat grafting or other medical procedures that include organ and tissue transplants resulting in increased viability of the graft with longer retention times. In an exemplary embodiment, the inventive powder composition of this application can be used as a tumescent base, and depending upon the formulation, can be reconstituted into a saline solution (including a saline-bag) or water-for-injection (WFI) at point of use. In other exemplary embodiments the sterilized powder formulation has long term stability, which increases production efficiency and reduces production costs.

An exemplary embodiment of this disclosure is directed to a powder formulation, comprising: reduced glutathione; L-ascorbic acid; a sugar; L-arginine; a buffering agent; and a physically acceptable salt, wherein the powder formulation when reconstituted at a point of use in a saline or sterile water media forms a reconstituted formulation having a pH of about 7.

Another exemplary embodiment is directed to a powder formulation, comprising: a reducing agent; an antioxidant; optionally, a sugar; optionally, a nitric oxide substrate; a buffering agent; and a physically acceptable salt, wherein the powder formulation when reconstituted at a point of use in a saline or sterile water media forms a reconstituted formulation having a pH of about 7. The reducing agent is at least one selected from reduced-glutathione and cysteinylglycine.

In another exemplary embodiment, the antioxidant is ascorbic acid.

In another exemplary embodiment, the powder formulation comprises the nitric oxide substrate, and wherein the nitric oxide substrate is L-arginine.

Another exemplary embodiment of this disclosure is directed to a method for preserving a tissue or organ, comprising: reconstituting the powder formulation described herein in a saline or sterile water media to form a reconstituted formulation; and bringing the tissue or organ into contact with the reconstituted formulation.

Another exemplary embodiment of this disclosure is directed to a method for preparing an organ or tissue preservation solution, comprising: the powder formulation described herein; reconstituting the powder formulation at point of use in a saline or a sterile water medium to form a reconstituted formulation; and contacting the organ or tissue with the reconstituted formulation.

In various exemplary embodiments, the sugar can be selected from the group consisting of glucose, fructose, mannose and ribose, and preferably the sugar can be D-glucose.

In various exemplary embodiments, the physically acceptable salt comprises: anhydrous calcium chloride; potassium phosphate monobasic; anhydrous magnesium sulfate; anhydrous magnesium chloride; anhydrous magnesium chloride; anhydrous sodium phosphate dibasic; anhydrous potassium chloride; and sodium bicarbonate.

In various exemplary embodiments, the reduced glutathione, L-ascorbic acid, the sugar and the L-arginine are included in the powder formulation in amounts that produce the reconstituted formulation having a concentration of the L-arginine from about 250 μM to about 2000 μM, a concentration of the sugar from about 5 mM-50 mM, a concentration of the L-glutathione from about 50 μM to about 3000 μM, and a concentration of the L-ascorbic acid from about 500 μM to about 4000 μM.

In various exemplary embodiments, the powder formulation does not include sodium chloride.

In various exemplary embodiments, the sterile water medium is a water-for-injection medium.

In various exemplary embodiments, the saline medium is a 0.9% sodium chloride solution.

In various exemplary embodiments, the tissue or organ is selected from the group consisting of fat or fat tissue grafts, saphenous veins, internal thoracic/mammary arteries, epigastric arteries, gastroepiploic arteries, radial arteries, heart, lungs, kidney, brain, muscle grafts, skin, intestine, bone, appendages, eyes, and portions of said tissue or organs.

Another exemplary embodiment of this disclosure is directed to a unit dosage form, comprising the powder formulation described herein.

In another exemplary embodiment, the unit dosage form is sterilized by gamma irradiation.

In another exemplary embodiment, the unit dosage form includes one or more components of the physically acceptable salt in separate packages.

In another exemplary embodiment, the one or more components are in solid or liquid forms.

Another exemplary embodiment of this disclosure is directed to a kit, comprising the unit dosage form described herein, and instructions for use.

It should be understood that the various individual aspects and features of the present invention described herein can be combined with any one or more individual aspect or feature, in any number, to form embodiments of the present invention that are specifically contemplated and encompassed by the present invention.

### BRIEF DESCRIPTION OF THE DRAWINGS

FIG. 1 is a flowchart of the cytotoxicity and lipid accumulation study design corresponding to the working Examples. The control is either a lactated ringers or saline solution;

FIG. 2 is a graphical representation of preadipocyte viability after 1, 5, and 24-hour treatments according to Experimental Example 1;

FIG. 3 is a graphical representation of mature Adipocyte (differentiated in vitro) viability after 1, 5, and 24-hour treatments according to Experimental Example 2;

FIG. 4 is a graphical representation of mature adipocyte lipid accumulation after 1, 5, and 24-hour treatments of preadipocytes prior to differentiation according to Experimental Example 3;

FIG. 5 is a graphical representation of pre-adipocyte viability after 1-hour, 5-hour and 24-hour treatments prior to differentiation according to Experimental Example 4;

FIG. 6 is a graphical representation of mature Adipocyte viability after 1, 5, and 24-hour treatment prior to differentiation according to Experimental Example 5;

FIG. 7 are fluoromicrograph mages showing live/dead stained adipocytes for counting of mature adipocytes and measuring viability of the differentiated adipocytes pre-differentiation according to Experimental Example 6;

FIGS. 8A and 8B are graphical representations of mature adipocyte cell number and viability respectively after 1, 5, and 24-hour treatments following differentiation according to Experimental Example 7;

FIG. 9 is a graphic representation of mature adipocyte (differentiated in vitro) viability after 1, 5, and 24-hour treatments post-differentiation according to Experimental Example 8;

FIG. 10 are flouromicrograph images showing live/dead stained adipocytes for counting of mature adipocytes and measuring viability of the differentiated adipocytes according to Experimental Example 8; and

FIG. 11 is a graphical representation of mature adipocyte lipid accumulation after 1, 5, and 24-hour post-differentiation treatment according to Experimental Example 8.

### DETAILED DESCRIPTION

Further aspects, features and advantages of this invention will become apparent from the detailed description which follows.

As used herein, the singular forms “a”, “an” and “the” are intended to include the plural forms as well, unless the context clearly indicates otherwise.

As used herein, “about” is a term of approximation and is intended to include minor variations in the literally stated amounts, as would be understood by those skilled in the art. Such variations include, for example, standard deviations associated with techniques commonly used to measure the amounts of the constituent elements or components of an alloy or composite material, or other properties and characteristics, up to and including a variation of ±10%. All of the values characterized by the above-described modifier “about,” are also intended to include the exact numerical values disclosed herein. Moreover, all ranges include the upper and lower limits.

Any compositions described herein are intended to encompass compositions which consist of, consist essentially of, as well as comprise, the various constituents identified herein, unless explicitly indicated to the contrary.

As used herein, the recitation of a numerical range for a variable is intended to convey that the variable can be equal to any value(s) within that range, as well as any and all sub-ranges encompassed by the broader range. Thus, the variable can be equal to any integer value or values within the numerical range, including the endpoints of the range. As an example, a variable which is described as having values between 0 and 10, can be 0, 4, 2-6, 2.75, 3.19-4.47, etc.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

As used herein, the term “patient” includes members of the animal kingdom including but not limited to human beings.

As used herein, “organ” includes, but is not limited to, the heart, veins, arteries, lungs, liver, pancreas and the kidneys. Portions of organs are also contemplated.

As used herein, “tissue” includes, but is not limited to mammalian tissue, including animal or human tissue, which can be connective, muscle, nervous, adipose, blood or epithelial tissues, and can include a single tissue or a collection of tissues that form a common function.

As used herein, “sterile water” includes, but is not limited to, (a) sterile water for injection, USP, (b) sterile distilled deionized water, and (c) sterile water for irrigation.

As used herein, “cardioplegia” includes, but is not limited to, paralysis of the heart.

As used herein, “moderate hypothermia” is about 10° C. to about 21° C.

As used herein, an “antioxidant” is a substance that, when present in a mixture or structure containing an oxidizable substrate biological molecule, delays or prevents oxidation of the substrate biological molecule. For example, ascorbic acid is an antioxidant.

As used herein, a “balanced salt solution” is defined as an aqueous solution that is osmotically balanced to prevent acute cell or tissue damage.

As used herein, a “buffered salt solution” is defined as a balanced salt solution to which chemicals have been added to maintain a predetermined physiological pH range.

As used herein, “GALA” or “GALA™ refers to a type of tissue preservation solution including glutathione, ascorbic acid, L-arginine and a balanced salt solution.

As used herein, “graft” is defined as tissue that is transplanted or implanted in a part of the body to repair a defect or to improve the appearance. A graft can be homologous (transplanted into the same patient from which it was originally removed) or heterologous (transplanted into a recipient different from the donor)

As used herein, “fat grafting” is defined as any surgical process that includes transferring fat or fat tissues from one area to another area of a mammalian body. In some cases, fat may be grafted back into the same area from which it was removed.

As used herein, a “harvested bypass conduit” is defined as a surgically installed alternate route for the blood to bypass an obstruction.

As used herein, a “solution of cardioplegia” is defined as a solution that aids in the preservation of the heart during transport or surgery.

As used herein, a “cellular reducing agent” is defined as a substance that loses electrons easily thereby causing other substances to be reduced chemically.

As used herein, a “buffering agent” is a chemical compound or compounds (generally a weak acid or base or combination thereof) that maintains the pH of a solution in a desired range. Examples of physiologically tolerated buffers are, for example, acetate, bicarbonate, citrate, sodium phosphate, succinate, histidine, sulfate, nitrate, and pyruvate buffers, but are not limited thereto.

As used herein, a “pH adjusting agent” is chemical compound that can raise or lower pH when added to a solution. Preferred pH adjusting agents include HCl for lowering pH and NaHCO₃ for raising pH, but are not limited thereto.

As used herein, an “isotonic solution” refers to a solution that has the same salt concentration as cells.

As used herein a “physiologic solution” is defined as an aqueous salt solution which is compatible with normal tissue, by virtue of being isotonic with normal interstitial fluids and having physiological pH.

As used herein, a “tumescent base” is defined as a fluid, including solutions, that can form the basis of a tumescent solution, i.e., typically normal saline, lactated ringers or similar used in liposuction and fat grafting and to which other components may be added based on surgeons' preferences (i.e., lidocaine for pain relief, epinephrine to reduce bleeding and bruising, and the like). Large volumes of this solution are injected subcutaneously to infiltrate fat tissue to facilitate liposuction aspiration using suction and aspiration cannulae. As an option, tumescent solutions may contain epinephrine and/or lidocaine.

As used herein, a “powder formulation” is defined as a dry, solid substance, composed of finely divided components (salts/organics) with or without excipients and intended for internal or external use. It is a solid substance in finely divided state typically obtained by crushing, grinding, sniffing and/or comminuting. In a preferred embodiment, the components of the powder formulation can be anhydrous.

As used herein, a “saline solution” is defined as an NaCl solution used in surgical and medical treatment processes.

As used herein, “sem” means standard error of the mean.

An exemplary embodiment of this disclosure is directed to a powder formulation, comprising: reduced glutathione; L-ascorbic acid; a sugar; L-arginine; a buffering agent; and a physically acceptable salt, wherein the powder formulation when reconstituted at a point of use in a saline or sterile water media forms a reconstituted formulation having a pH of about 7.

Another exemplary embodiment is directed to a powder formulation, comprising: a reducing agent; an antioxidant; optionally, a sugar; optionally, a nitric oxide substrate; a buffering agent; and a physically acceptable salt, wherein the powder formulation when reconstituted at a point of use in a saline or sterile water media forms a reconstituted formulation having a pH of about 7. The reducing agent is at least one selected from reduced-glutathione and cysteinylglycine.

In another exemplary embodiment, the antioxidant is ascorbic acid.

In another exemplary embodiment, the powder formulation comprises the nitric oxide substrate, and wherein the nitric oxide substrate is L-arginine.

In another exemplary embodiment, the powder formulation does not include the nitric oxide substrate. In yet another embodiment, the powder formulation does not include L-arginine.

In another exemplary embodiment, the powder formulation comprises reduced glutathione; L-ascorbic acid; the sugar, which is D-glucose; a buffering agent; and a physically acceptable salt, wherein the powder formulation does not include a calcium salt and sodium chloride.

In another exemplary embodiment, the powder formulation does not include sodium chloride.

In another exemplary embodiment, the powder formulation does not include a calcium salt.

In another exemplary embodiment, the powder formulation does not include arginine and calcium salt.

In another exemplary embodiment, the powder formulation does not include arginine and sodium chloride.

In another exemplary embodiment, the powder formulation does not include sodium chloride, arginine and calcium salt. Another exemplary embodiment of this disclosure is directed to a method for preserving a tissue or organ, comprising: reconstituting the powder formulation described herein in a saline or sterile water media to form a reconstituted formulation; and bringing the tissue or organ into contact with the reconstituted formulation.

Another exemplary embodiment of this disclosure is directed to a method for preparing an organ or tissue preservation solution, comprising: the powder formulation described herein; reconstituting the powder formulation at point of use in a saline or a sterile water medium to form a reconstituted formulation; and contacting the organ or tissue with the reconstituted formulation.

In various exemplary embodiments, the sugar can be selected from the group consisting of glucose, fructose, mannose and ribose, and preferably the sugar can be D-glucose.

In various exemplary embodiments, the physically acceptable salt comprises: anhydrous calcium chloride; potassium phosphate monobasic; anhydrous magnesium sulfate; anhydrous magnesium chloride; anhydrous magnesium chloride; anhydrous sodium phosphate dibasic; anhydrous potassium chloride; and sodium bicarbonate.

In various exemplary embodiments, the reduced glutathione, L-ascorbic acid, the sugar and the L-arginine are included in the powder formulation in amounts that produce the reconstituted formulation having a concentration of the L-arginine from about 250 μM to about 2000 μM, a concentration of the sugar from about 5 mM-50 mM, a concentration of the L-glutathione from about 50 μM to about 3000 μM, and a concentration of the L-ascorbic acid from about 500 μM to about 4000 μM.

In various exemplary embodiments, the powder formulation does not include sodium chloride.

In various exemplary embodiments, the sterile water medium is a water-for-injection medium.

In various exemplary embodiments, the saline medium is a 0.9% sodium chloride solution.

In various exemplary embodiments, the tissue or organ is selected from the group consisting of fat or fat tissue grafts, saphenous veins, internal thoracic/mammary arteries, epigastric arteries, gastroepiploic arteries, radial arteries, heart, lungs, kidney, brain, muscle grafts, skin, intestine, bone, appendages, eyes, and portions of said tissue or organs.

Another exemplary embodiment of this disclosure is directed to a unit dosage form, comprising the powder formulation described herein.

In another exemplary embodiment, the unit dosage form is sterilized by gamma irradiation.

In another exemplary embodiment, the unit dosage form includes one or more components of the physically acceptable salt in separate packages.

In another exemplary embodiment, the one or more components are in solid or liquid forms.

Another exemplary embodiment of this disclosure is directed to a kit, comprising the unit dosage form described herein, and instructions for use.

As described above, the stability of standard GALA™ solutions can be improved by separating the formulation into a first Solution A including a balanced solution and having a pH of at least 7, and a second Solution B, which includes water, ascorbic acid, L-glutathione, L-arginine and D-glucose and has a pH of less than 7 (“stable two-solution formulation”). This stable two-solution formulation, commercially available as DuraGraft™, is currently manufactured as a two-solution-containing kit with an aseptically manufactured Solution A bottle and Solution B vial containing two separate solutions. Solutions A and B are mixed at the point-of-use (POU) to generate an organ and tissue preservation solution. However, manufacture of the two-solution kit requires two separate and costly GMP manufacturing runs—one for Solution A and one for Solution B. Additionally, Solution B cannot be terminally sterilized and therefore the Sterility Assurance Level (SAL) of the two solution-containing formulation cannot go below 10⁻³, making the product not viable for indications requiring a product with a lower SAL. The powder formulation described in this application addresses both these issues by requiring only one GMP run rather than multiple runs, thereby reducing production costs. Additionally, the powder formulation can be terminally sterilized allowing the SAL of the product to be reduced to 10⁻⁶, resulting in a product with decreased bioburden risk as well as generating a product that can be used in indications requiring a SAL of 10⁻⁶. For example, such powder formulations can be used as a tumescent base for various surgical procedures, including fat grafting. Additionally, the GMP manufacturing runs would not need to involve aseptic processing because of the terminal sterilization, further reducing production costs. The terminal sterilization can include gamma irradiation of the packaged powder formulation, but the sterilization process is not limited thereto, and any suitable terminal sterilization procedures can be used. Additionally, a small vial of powder formulation can ultimately be reconstituted into very large volumes (for e.g., 3 L of liquid product) of tumescent fluid required during fat grafting and other medical and/or surgical procedures. Thus, space saving feature is an added benefit associated with the powder formulation, and the powder formulation can be easily stored in an OR storage space. In some exemplary embodiments, the powder formulation may be initially reconstituted into a small volume of sterile water followed by reconstitution in saline solution. In an exemplary embodiment, the powder formulation may be initially reconstituted in about 5 ml to about 300 ml of sterile water, preferably about 10 ml to about 200 ml, about 15 ml to about 100 ml, about 20 ml to about 50 ml and the like, followed by reconstitution in saline solution. The volume of the sterile water used in the initial reconstitution step is not limited hereto, and can include any values within any of the recited ranges and/or combinations thereof. The components of the powder formulations described herein are included in their anhydrous forms, but are not limited thereto, and were developed for reconstitution at point of use using a saline solution, a salt solution and/or a water-for-injection (WFI) medium.

In an exemplary embodiment, the powder formulation includes reduced glutathione; a reducing agent such as ascorbic acid; a sugar such as D-Glucose; a nitric oxide substrate such as L-arginine; a buffering agent, and a physically acceptable salt. In another exemplary embodiment, cysteinylglycine can be substituted for reduced glutathione or a mixture of cysteinylglycine and reduced glutathione can be present in the powder formulation. Any suitable sugar can be used in place of glucose, but preferably any monosaccharide including but not limited to fructose, mannose and ribose can be used. In another exemplary embodiment, the powder formulation can include a reduced glutathione, a reducing agent, a buffering agent, and a physically acceptable salt. In other exemplary embodiments, the powder formulation can exclude one or more of the sugar and/or arginine.

Physically acceptable salts are those that are capable of forming a balanced salt solution when reconstituted in an aqueous medium. A typical balanced salt solution generally comprises water, calcium ions, chloride ions, potassium ions, phosphate ions, magnesium ions and sodium ions. The physically acceptable salts can be any one or more selected from among: calcium chloride dihydrate, potassium chloride, potassium phosphate monobasic, magnesium chloride hexahydrate, magnesium sulfate heptahydrate, sodium chloride, sodium bicarbonate, sodium phosphate dibasic heptahydrate, calcium gluconate, calcium citrate, calcium carbonate, calcium lactate, calcium saccharate, and combinations thereof. The physically acceptable salts are not limited to those listed herein, and can include any suitable physically acceptable salt, including but not limited to salts of weak acids such as phosphoric acid, propionic acid, lactic acid, gluconic acid, and the like. In an exemplary embodiment, the physically acceptable salt can be one or more calcium salts of a weak acid. The physically acceptable salts are not limited, and can be included in their anhydrous forms or in their various hydrated forms. In a preferred exemplary embodiment, the physically acceptable salts are included in their anhydrous forms. In an alternate exemplary embodiment, the physically acceptable salts can be in their hydrated forms, including any degree of hydration, such as dihydrates, trihydrates, hexahydrates, heptahydrates, and the like. The salts in the solution are intended for buffering (to maintain pH) and to maintain isotonicity with respect to vascular conduits and other tissues and to maintain ionic balance. When reconstituted at a point of use, the physically acceptable salts will form a balanced salt solution at a concentration that will result in an isotonic solution. In an exemplary embodiment, the physically acceptable salts are included in the powder formulation in amounts that, when reconstituted in 1125 ml of saline solution or WFI medium, produce a balanced salt solution including about 0.14 grams/liter calcium chloride dihydrate, about 0.4 grams/liter potassium chloride, 0.06 grams/liter potassium phosphate monobasic, about 0.1 grams/liter magnesium chloride hexahydrate, about 0.1 grams/liter magnesium sulfate heptahydrate, about 8 grams/liter sodium chloride, about 0.36 grams/liter sodium bicarbonate, and about 0.03 grams/liter sodium phosphate dibasic heptahydrate. This is an exemplary reconstituted formulation, and other exemplary formulations can independently include other hydrated or anhydrous forms of the various components of the powder formulation, can exclude one or more components, and/or one or more components, in powder or liquid form, can be added to the powder formulation during reconstitution. In another exemplary embodiment, the physically acceptable salts are included in the powder formulation in amounts such that the pH of the reconstituted powder formulation at point of use can be about 7.0. In some embodiments, the pH can be about 7.3±0.4. The pH at the point of use can be adjusted using any known pH adjusting agents. Preferred pH adjusting agents are 4N HCl to decrease the pH and 84% NaHCO3 to increase the pH.

Optionally, the powder formulation can include non-steroidal anti-inflammatory agents such as aspirin, naproxen and ibuprofen, or can be combined with such non-steroidal anti-inflammatory agents when reconstituted at point of use. Optionally, local anesthesia medications can be added at the point of use. Examples of such local anesthesia medications include but are not limited to lidocaine, articaine, mepivacaine, bupivacaine, ropicavaine and chloroprocaine, and any other suitable anesthesia medications known in the art. Optionally, the powder formulation can also include epinephrine or other suitable medication to reduce intraoperative blood loss, and/or these additional components can be added at point of use.

The organic components are intended to maintain additional buffering capability, osmolality, to provide a non-oxidizing environment for tissues, organs and cells, and when arginine is present, to maintain NO levels and prevent storage lesions in some tissues (i.e., vascular grafts) when that tissue is outside the body. The four organic components: L-glutathione, L-ascorbic acid, L-arginine and D-glucose, are normal constituents of blood and are also included for their roles in preserving and maintaining the extracellular environment of vascular conduits.

L-glutathione and L-ascorbic acid are antioxidants that prevent oxidative damage to cells by reacting with or neutralizing free radicals. The functions of these antioxidants in an organ or tissue preservation solution are: 1) to stabilize other components of the solution by preventing oxidation, thereby improving the stability and shelf life of product; and 2) to prevent oxidative damage to cell membranes, cellular components and extracellular matrix structures. Oxidative damage has been shown to damage the structural integrity of the extracellular architecture, thereby causing an interruption in the endothelial cell lining. Oxidative damage is also the basis for reperfusion injury that occurs following transplantation. Reperfusion injury exacerbates damage to tissue and organs. The sugar in the formulation is intended to provide an energy source to support anaerobic metabolism for the tissue, cell or organ under ischemic/hypoxic conditions.

In an exemplary embodiment, the powder formulation includes potassium chloride, potassium phosphate, magnesium chloride, magnesium sulfate, sodium bicarbonate, sodium phosphate, reduced L-glutathione, D-glucose, L-arginine and L-ascorbic acid. In another exemplary embodiment, the powder formulation includes potassium chloride, potassium phosphate monobasic, magnesium chloride hexahydrate, magnesium sulfate heptahydrate, sodium bicarbonate, sodium phosphate dibasic, reduced L-glutathione, D-glucose, L-arginine and L-ascorbic acid. In another exemplary embodiment, the powder formulation includes potassium chloride, potassium phosphate, magnesium chloride, magnesium sulfate, sodium bicarbonate, sodium phosphate, reduced L-glutathione, D-glucose, L-arginine and L-ascorbic acid. In a preferred embodiment, the powder formulation does not include sodium chloride (NaCl) prior to reconstitution. In another exemplary embodiment, the powder formulation does not include a calcium salt prior to reconstitution. In another exemplary embodiment, the powder formulation does not include arginine prior to reconstitution. In another exemplary embodiment, the powder formulation does not include arginine and a calcium salt prior to reconstitution. In another exemplary embodiment, the powder formulation does not include arginine and sodium chloride prior to reconstitution. In another exemplary embodiment, the powder formulation does not include sodium chloride, arginine and a calcium salt prior to reconstitution. In another exemplary embodiment, one or more of the components of the powder formulation may be added as a liquid during the manufacturing process and/or one or more of the components of the powder formulation may be provided as a liquid formulation that is added at the time of reconstitution. In yet another exemplary embodiment, calcium chloride, or any suitable calcium salt, is added as a separate liquid component that is added during reconstitution.

In an exemplary embodiment, the amount of potassium chloride is about 0.45 gm to about 0.45 gm, potassium phosphate is about 0.07 gm to about 0.07 gm, magnesium chloride hexahydrate is about 0.11 gm to about 0.11 gm, magnesium sulfate heptahydrate is about 0.11 gm to about 0.11 gm, sodium bicarbonate is about 0.40 gm to about 0.40 gm, sodium phosphate is about 0.03 gm, reduced L-glutathione is about 0.35 gm to about 0.35 gm, D-glucose is about 1.12 gm to about 1.12 gm, L-arginine is about 0.175 gm to about 0.17 gm and L-ascorbic acid is about 0.10 gm to about 0.10 gm. In another exemplary embodiment, the amount of potassium chloride is about 0.45 gm to about 0.45 gm, potassium phosphate is about 0.07 gm to about 0.07 gm, magnesium chloride is about 0.05 gm, magnesium sulfate is about 0.06 gm, sodium bicarbonate is about 0.40 gm to about 0.40 gm, sodium phosphate is about 0.03 gm, reduced L-glutathione is about 0.35 gm to about 0.35 gm, D-glucose is about 1.12 gm to about 1.1 gm, L-arginine is about 0.17 gm to about 0.17 gm and L-ascorbic acid is about 0.10 gm to about 0.10 gm. The amount(s) of the various components are not limited hereto, and can include any values within any of the recited ranges and/or combinations thereof. The powder formulation of this exemplary embodiment can be reconstituted in 1125 ml of sterile water or saline solution at point of use.

When reconstituted, the concentration of L-arginine should be from about 250 μM to about 2000 μM, glucose should be about 5 mM to about 50 mM, L-glutathione should be about 50 μM to about 3000 μM, and L-ascorbic acid should be about 500 μM to about 4000 μM. The concentration of L-arginine can be from about 300 μM to about 1750 μM, about 350 μM to about 1500 μM, about 400 μM to about 1250 Mm, about 500 μM to about 1000 μM, and the like. The concentration of the glucose can be from about 5.1 mM to about 45 mM, about 5.2 mM to about 40 mM, about 5.3 mM to about 35 mM, about 5.4 mM to about 30 mM, about 5.5 mM to about 25 mM, about 5 mM to about 20 mM, and the like. The concentration of the L-glutathione can be from about 50 μM to about 2500 μM, about 60 μM to about 2000 μM, about 65 μM to about 1500 μM, about 70 μM to about 1000 μM, and the like. The concentration of the L-ascorbic acid can be about 550 μM to about 3500 μM, about 600 μM to about 3000 μM, about 650 μM to about 2500 μM, about 700 μM tot about 2000 μM, and the like. The amount(s) of the various components are not limited hereto, and can include any values within any of the recited ranges and/or combinations thereof.

In another exemplary embodiment, the powder formulation can optionally include an anticoagulant in an amount sufficient to help prevent clotting of blood within the vasculature of a tissue or organ. The anticoagulant can also be added during reconstitution at point of use. Exemplary anticoagulants include heparin and himdin, but other anticoagulants such as aspirin can be used. An exemplary embodiment includes heparin in concentration ranges from about 50 units/mL to about 250 units/mL.

In another exemplary embodiment, the powder formulation can optionally include erythropoietin (EPO) or EPO can be added at point of use. If EPO is added, either in the powder formulation or at point of use, the final EPO concentration should be about 5 units/mL.

Generally, the powder formulation will not include any organic components other than those specifically listed, as these would be unnecessary and increase production costs. In particular, organic compounds other than those previously listed which are found in other types of tissue preservation solutions, would not be included in the powder formulation, to minimize cost, which could be detrimental to the final product. However, any additional components that do not materially affect the basic characteristics of the powder formulation can be included. Other organics can be added to the reconstituted formulation per the surgeons' preference.

The solutions, devices, and perfusion methods of the present invention are not limited to use with a particular tissue, organ or cell type. For example, the invention can be used with harvested saphenous veins, internal thoracic/mammary arteries, epigastric arteries, gastroepiploic arteries and radial arteries used in coronary bypass grafting (CABG), adipose tissue used in fat grafting, and the like. The present invention can also be used to maintain organs and tissue during transplant operations. The present invention is not limited to any particular tissue or organ. For example, it is contemplated that such organs or tissues can be heart, lungs, kidney, brain, blood, platelets, stem cells, muscle grafts, skin, intestine, bone, appendages, eyes, and the like, or portions thereof. Additionally, it is contemplated that the reconstituted formulation of the present invention can be used to wash and bathe tissues and organs that have not been removed from the patient. For example, it is contemplated that the present invention be used during cardioplegia. It is also contemplated that the present invention be used in, for example, emergency procedures where a tissue or organ may need to be bathed to preserve it until surgery or other medical attention can be obtained. In this regard, the powder formulation can be made available to emergency medical personnel both in hospital settings and “in the field” (i.e., in ambulances or in temporary emergency medical facilities), and can be easily reconstituted at point of use in saline or WFI media.

Reconstituted powder formulations can also be used as a lavage-solution in surgeries, in particular plastic or reconstructive surgery. This includes but is not limited to liposuction, fat grafting, breast reconstruction, mastectomy or other operations in which an isotonic, pH-balanced solution, such as that of the present invention, would be desirable. A reconstituted powder formulation can also be used to flush or prime a dialysis machine using the manufacturer's procedure for priming or flushing, or used in conjunction with a venous external support.

The powder formulation can be packaged in a glass vial, such as a borosilicate glass vial, which demonstrates the feasibility of long-term storage at room temperature.

The principles of the present invention, as well as certain exemplary features and embodiments thereof, will now be described by reference to the following non-limiting examples.

An exemplary embodiment of the subject application is directed to the manufacture of a powder formulation comprising: L-glutathione, D-glucose, L-arginine, L-ascorbic acid, and additive components that form a balanced salt solution when reconstituted at point of use. The additive components can be selected from among calcium chloride, potassium phosphate, magnesium sulfate, magnesium chloride, sodium phosphate, potassium chloride and sodium bicarbonate. In a preferred embodiment, each of the components of the powder formulation are in their anhydrous forms, but the powder formulations are not limited thereto, and hydrated forms of the components can also be included in the powder formulations to the extent that they do not affect the physiological properties of the powder formulation prior to and after reconstitution. In a preferred embodiment, the powder formulation includes: L-glutathione, D-glucose, L-arginine, L-ascorbic acid, calcium chloride anhydrous, potassium phosphate monobasic, magnesium sulfate anhydrous, magnesium chloride anhydrous, magnesium chloride anhydrous, sodium phosphate dibasic anhydrous, potassium chloride, and sodium bicarbonate.

An exemplary process for manufacturing powder formulations is described herein, but this process should not be considered to be a limiting disclosure, and the process can be subject to various parameter modifications as required to achieve the inventive aspect of the powder formulations of this application. The exemplary process for manufacturing powder formulations according to an exemplary embodiment includes: a first step of screening components of the powder formulation including L-glutathione, D-glucose, L-arginine, L-ascorbic acid, calcium chloride anhydrous, potassium phosphate monobasic, magnesium sulfate anhydrous, magnesium chloride anhydrous, magnesium chloride anhydrous, sodium phosphate dibasic anhydrous by passing through a mesh screen to yield components within a targeted particle size. As an example, a #30 mesh screen can be used to remove particles larger than 595 μm, or a #35 mesh screen to remove particles larger than 500 μm, or a #40 mesh screen to remove particles larger than 400 m. After the screening process is completed, the screened components are mixed or blended together. A second optional step of screening additional components of the powder formulation including potassium chloride and sodium bicarbonate by passing through a #30 mesh screen, #35 mesh screen, or #40 mesh screen can be completed. Typically, but not always, the same mesh size is used for all of the components to minimize particle segregation. In another exemplary embodiment, the first and second steps can be performed simultaneously. In another exemplary embodiment, each of the components, or any combination thereof, can be screened in separate steps prior to mixing. In the examples described herein, the mixed components of the powder formulation were milled using a Fritz Mill (Screen #1722-0020) at 6000 rpm with knives forward. The milled mixture can be loaded into glass vials, closed with a rubber stopper and secured with a flip-off/tear-off combination seal. This exemplary process can include an optional step of milling one or more of the components of the powder formulation prior to an initial step of screening the various components using a mesh screen, as described above.

### Terminal Sterilization Testing

Following the preparation of a powder formulation, terminal sterilization by gamma irradiation was carried out to achieve greater Sterility Assurance Level (SAL, <10⁻⁶).

### Preparation Method A—Powder Formulations for Gamma Radiation Studies

Components of the various powder formulations are shown in Table 1. All the components in the amounts listed in Table 1 were blended to form a uniform powder mixture. The final blend size including all the components was about 120 grams. This process was repeated twice more, and the blended mixture of all three preparation steps were mixed to form a final blended mixture having a blend size of about 360 grams, and the final blended mix was transferred to a container. The final blended mix was mixed gently by shaking the container, and then dispensed into separate bags and vacuum sealed. For testing, a required quantity of the finally blended mixture was transferred to a 20 mL borosilicate glass vial, which was immediately capped with a bromobutyl rubber stopper and secured with a crimp cap.

TABLE 1

| Ingredient | Prep. | Prep. | Prep. | Prep. | Prep. | Prep. |
|---|---|---|---|---|---|---|
| (g/1125 mL) | Ex. A1 | Ex. A2 | Ex. A3 | Ex. A4 | Ex. A5 | Ex. A6 |
| Potassium Chloride | 0.4504 | 0.4500 | 0.4500 | 0.4500 | 0.4500 | — |
| Potassium phosphate | 0.0674 | 0.0675 | 0.0675 | 0.0675 | 0.0675 | — |
| monobasic | | | | | | |
| Magnesium chloride | 0.1121 | — | 0.1125 | 0.1125 | 0.1125 | — |
| hexahydrate | | | | | | |
| Magnesium sulfate | 0.1137 | — | 0.1126 | 0.1126 | 0.1126 | — |
| heptahydrate | | | | | | |
| Magnesium chloride | — | 0.0527 | — | — | — | — |
| Magnesium sulfate | — | 0.0550 | — | — | — | — |
| sodium bicarbonate | 0.4054 | 0.4050 | 0.4050 | 0.4050 | 0.4050 | — |
| Sodium phosphate | 0.0303 | 0.0303 | 0.0303 | 0.0303 | 0.0303 | — |
| dibasic, anhydrous | | | | | | |
| L-glutathione | 0.3492 | 0.3489 | 0.3489 | 0.3489 | — | 0.3489 |
| reduced | | | | | | |
| D-glucose | 1.1248 | 1.1251 | 1.1251 | 1.1251 | — | 1.1251 |
| L-arginine | 0.1685 | 0.1687 | — | 0.1687 | 0.1687 | — |
| L-ascorbic acid | 0.1011 | 0.1012 | 0.1012 | 0.1012 | — | 0.1012 |
| Amount of powder | 2.9229 | 2.8044 | 2.7530 | 2.9218 | 1.3466 | 1.5752 |
| for reconstitution (g) | | | | | | |
| Packaging for | Ambient | Vacuum | Vacuum | Vacuum | Vacuum | Vacuum |
| Gamma study | atmosphere | | | | | |

### Preparation Method B—Liquid Formulations for Comparative Gamma Radiation Studies (Control)

Liquid formulations were prepared as control formulations to assess differences in stability profiles of the powder formulation and the control post and pre irradiation. Liquid formulations shown in Table 2 were prepared under a biological safety cabinet in a 2000 mL (2 liters) batch size. A required quantity of water-for-injection (WFI) was transferred to a clean 3-neck bottle and purged with Argon gas for an hour using sterile silicone tubing attached to a 0.2-μm sterile filter. A clean stir bar was added to the container, and the water was stirred at 350-550 rpm. The components of the liquid formulations, as listed in Table 2, were added one by one using a glass funnel while purging the solution with Argon gas. After all components were dissolved completely, the solution was divided into three portions, and the pH of each individual portion was adjusted to about 7.35 to about 7.45 using 1 μM NaOH, a pH of about 8.0 using 1 μM NaH, and a pH of about 3.0 using 1 μM HCl. About 18-19 mL of each portion was transferred into individual 20 ml sterile vials, Argon gas was added to the headspace in the vials, and the vials were sealed with rubber stoppers and secured with aluminum crimp caps. The vials were stored at 2° C. to 8° C.

TABLE 2

| | Prep. Ex. B1: | | Prep. Ex. B2: | | Prep. Ex. B3: | |
|---|---|---|---|---|---|---|
| | Solution A + B* | | Solution A^{†} | | Solution B^{†} | |
| | g/125 | | g/65 | | g/60 | |
| Ingredients | mL | g/L | mL | g/L | mL | g/L |
| Potassium chloride | 0.4500 | 3.5997 | 0.4500 | 6.9225 | — | — |
| Potassium phosphate | 0.0675 | 0.5401 | 0.0675 | 1.0387 | — | — |
| monobasic | | | | | | |
| Magnesium chloride | 0.1125 | 0.9002 | 0.1125 | 1.7312 | — | — |
| hexahydrate | | | | | | |
| Magnesium sulfate | 0.1126 | 0.9006 | 0.1126 | 1.7320 | — | — |
| heptahydrate | | | | | | |
| sodium bicarbonate | 0.4050 | 3.2397 | 0.4050 | 6.2302 | — | — |
| Sodium phosphate | 0.0303 | 0.2427 | 0.0303 | 0.4668 | — | — |
| dibasic, anhydrous | | | | | | |
| L-glutathione | 0.3489 | 2.7908 | — | — | 0.3489 | 5.8142 |
| reduced | | | | | | |
| D-glucose | 1.1251 | 9.0004 | — | — | 1.1251 | 18.751 |
| L-arginine | 0.1687 | 1.3499 | — | — | 0.1687 | 2.812 |
| L-ascorbic acid | 0.1012 | 0.8100 | — | — | 0.1012 | 1.687 |

*Solution A + B is 8x DuraGraft ™ which is mixed with 1000 mL Saline to represent 1x DuraGraft ™
^{†}65 mL of Solution-A is mixed with 60 mL of Solution-B and 1000 mL of Saline to represent 1x DuraGraft ™

### EXAMPLES

The powder blends Preparation Examples A1 to A6 described in Table 1 were prepared according to the method described in Preparation Method A as working examples. The powder blend for each example was dissolved in 125 mL of WFI and then reconstituted with 1-liter of saline. The reconstituted powder formulations in saline of the working examples were subjected to gamma irradiation conditions at 15 kGy and 25 kGy.

### Comparative Examples

Liquid formulations of Preparation Examples B1 to B3 as described in Table 2 were prepared according to the method described in Preparation Method 2 as comparative examples. The liquid formulations of the comparative examples were also subjected to gamma irradiation conditions at 15 kGy and 25 kGy.

### Gamma Irradiation Testing Protocol

Gamma irradiation testing was carried out in an ExCell® Irradiator manufactured by Sterigenics International Inc. (Oak Brook, IL). The number of vials and number of pouches were selected: 1) to achieve about 0.15 g/mL target density of a 10 inch by 10 inch by 10 inch carboard box that was placed inside the ExCell® Irradiator and exposed to Gamma radiation; and 2) to perform analytical testing at different stability timepoints. Irradiation of all formulations was carried out at 15 kGy and 25 kGy. Preliminary two-month accelerated and real-time stability studies were conducted on powder and liquid formulations following gamma irradiation (using 15 kGy and 25 kGy). Of the powder formulations, compositions included various hydration forms of the salts (i.e., some formulations used only anhydrous salts and others used salts of different hydrations forms). Gamma radiation did not impact the stability of the powder formulations, but greatly reduced stability of the organic components in the liquid formulations. Substantial reductions in stability were also observed for the liquid formulations independent of gamma irradiation. Of the powder formulations tested, there was some variation in the stability profiles (within various formulations) but the overall stability was substantially higher than the control liquid formulations which did not receive gamma irradiation. The projected stability data from these studies shows shelf-stability of more than 3 years at 25° C.

### Cytotoxicity and Lipid Accumulation Studies

Studies were conducted to evaluate effect of exposure to reconstituted powder formulation versus saline and lactated ringers-based tumescent fluid on human adipose tissue collected from women under an open Institutional Review Board (IRB). Viability and functionality assessments (lipid accumulation, ability to differentiate) were performed for surviving mature adipocytes and pre-adipocytes in the lipoaspirates, pre-adipocytes in expansion cultures derived from lipoaspirates, and mature adipocytes differentiated in vitro. Lipid accumulation data was also collected as a parameter for functioning mature adipocytes. Studies utilized reconstituted powder formulations as tumescent base for all studies described herein. Study participants included female subjects ranging in age from 34 to 45 years, having a body mass index (B.M.I.) of about 25 to 30, and the fat was harvested from the abdomen, hips and/or flank of study subjects. The culture media for adipocytes and preadipocytes is described in Table 3:

TABLE 3
Culture Media

| Preadipocyte Medium | Differentiation Medium | Maintenance medium |
|---|---|---|
| DMEM/Ham's F-12 | DMEM/Ham's F-12 | DMEM/Ham's F-12 |
| (1:1, v/v) | (1:1, v/v) | (1:1, v/v) |
| HEPES Buffer | HEPES Buffer | HEPES Buffer |
| Fatal Bovine Serum | Fatal Bovine Serum | Fatal Bovine Serum |
| | (FBS) | |
| Antibodies | Biotin | Biotin |
| | Pantothenate | Pantothenate |
| | Human insulin | Human insulin |
| | Dexamethasone | Dexamethasone |
| | Isobutylmethylxanthine | Antibodies |
| | (IBMX) | |
| | PPARy agonist | |
| | Antibodies | |

DMEM: Dulbecco's Modified Eagle's Medium;
HEPES: 4-(2-hydroxyethyl)-1-piperazineethanesulfonic acid

The assays conducted studied the cytotoxic and cytoprotective effects over 24 hours of exposure to the reconstituted powder formulation compared to standard saline-based or lactated-ringers-based tumescent fluid on primary adipocytes (floaters) isolated from fresh lipoaspirate. In addition, preadipocytes isolated from the same donor were isolated, cultured and treated with the reconstituted powder formulation and tumescent control for varying times up to 24 hours and tested for cytotoxicity or cytoprotection prior to differentiation. For differentiation studies, two weeks post-differentiation medium addition, the mature adipocytes were exposed to the reconstituted powder formulation or standard tumescent solution and tested for effects on viability as well as total lipid accumulation. Preadipocytes exposed to the reconstituted powder formulation or standard tumescent solution pre-differentiation treatment were also assessed for their ability to differentiate in mature adipocytes. Cytotoxicity was measured using the Cell Titer Blue Assay (Promega™) or the Dual Live/Dead Assay (Calcein AM and ethidium homodimer/Molecular Probes., Eugene OR). The amount of lipid accumulation was determined using ZenBio™ Total Triglyceride Assay and the test formulation and control were compared against each other and to vehicle controls. Additional details regarding the study protocols and results obtained are described below.

### Cytotoxicity and Lipid Accumulation Study Design

Fresh lipoaspirate was used to isolate mature adipocytes. On day 1, mature adipocytes were isolated and exposed to treatment with reconstituted powder formulation for 1, 5 and 24 hours. The cell concentrations were titrated to prevent Cell Titer Blue (CTB) signal saturation. CTB was added for one hour, and media was collected and its toxicity was measured at 1 hour and 5 hours. On day 2, CTB was added at 24 h from study initiation for one hour, and the media collected to measure its toxicity.

Preadipocyte Viability and Functionality Testing: In a concurrent study, preadipocytes were isolated according to the ZenBio™ protocol on day 1 and exposed to the reconstituted powder formulation or control. Alternatively, preadipocytes were expanded and cultured in flasks at 37° C. in preadipocyte media (PM1), and then the following protocol was carried out on the cultured media:
Day 2—cells were fed with fresh media.
Day 5—cells were passaged to a new flask.
Day 9—cells were seeded into a 96 well plate for experimentation.
Day 10—plates of expanded cells were treated with reconstituted powder formulation or saline-based tumescent solution at 0, 1, 5 and 24 hours. Alternatively, some plates of expanded cells were maintained in culture media until day 11.
Day 11—half of the plates cells that were exposed to the reconstituted powder formulation or control tumescent solution, were treated with CTB for 1 hour and then the cytotoxicity was measured. The other half of expanded cells that were exposed to the reconstituted powder formulation or control tumescent solution were not treated with CTB but were instead subjected to adipocyte differentiation protocol for two weeks. Expanded cells not exposed to the reconstituted powder formulation or control tumescent solution and not treated with CTB were subjected to the adipocyte differentiation protocol (naïve expanded preadipocytes)
Day 18—cell media was exchanged with maintenance media for all cells undergoing differentiation.
Day 25—differentiated cells (from non-naïve preadipocyte cells only) were exposed to CTB for 1 hour and toxicity was measured, following which, cells were lysed and total triglyceride content was measured. Cells differentiated from the naïve preadipocyte cultures were exposed to the reconstituted powder formulation or control tumescent solution for 1, 5 or 24 hours and then subjected to viability analysis and total lipid accumulation functional assays.

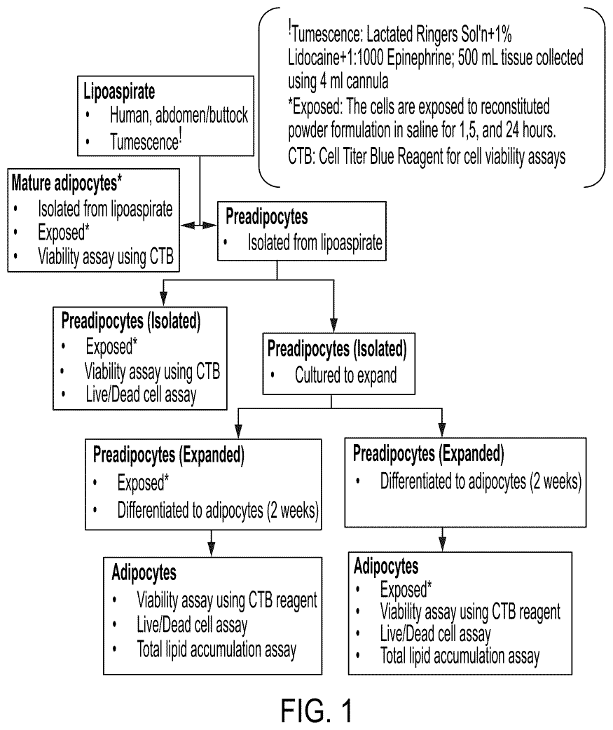

A flowchart of the cytotoxicity and lipid accumulation study design is also illustrated in FIG. 1.

### General Protocol for Preadipocyte Isolation

250 ml of fresh lipoaspirate was washed with 250 ml of phosphate buffered saline (PBS), and the washing was repeated five times. Following the washing step, cells were dissociated with collagenase for 15 minutes. After digestion, floaters were removed and preadipocytes were washed with an equal volume of PBS. The cells were pelleted and PBS and collagenase were removed, following by washing the cells with 50 ml of PBS. PBS was removed and cells were resuspended in preadipocyte growth media and plated into culture flasks. After 24 hours, the media was removed, and the cells were washed twice with PBS (2×) and replenished with fresh preadipocyte media. Cells were cultured for 4 days in PM1 and passaged. After 9 days, cells were split into 96-well plates for experimentation.

For preadipocyte exposure experiments: The day after the cells were collected for experimentation, the preadipocyte medium was removed and replaced with the reconstituted powder formulation or saline solution and exposed to treatment conditions (exposure to reconstituted powder formulation and saline) for 1, 5 and 24 hours. Viability was measured at 1, 5, and 24 hours either by CTB or Live Dead Assay.

For CTB assays: CTB was added to each treatment 1 hour prior to harvest. CTB was read directly in plate using a fluorescence reader set to 560ex/590em with 570 cutoff. The medium was removed, the cells were washed twice with PBS (1×), and 150 μl of differentiation media was added to each well. After an additional seven days, 90 μl of differentiation medium was removed and replaced with 120 μl of adipocyte maintenance medium. After seven additional days, the cells were prepared for assay by adding CTB to each treatment 1 hour prior to harvest. CTB was read directly in plate using fluorescence reader set to 560ex/590em with 570 cutoff. The medium was removed and the cells were washed twice with PBS (1×).

Manufacturer instructions were followed for Live Dead assays (Calcein AM and ethidium homodimer/Molecular Probes., Eugene OR).

### Lipogenesis Protocol

Lipid accumulation was studied following the ZenBio™ protocol. The absorbance values of the unknown samples were converted to glycerol concentrations by correlation to the glycerol concentration standard curves using the generated linear regression equation.

### Experimental Example 1

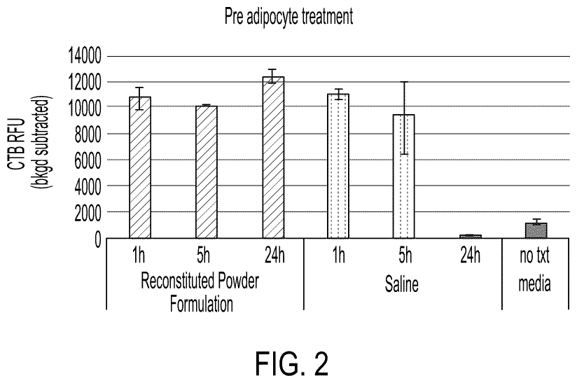

Preadipocyte viability after 1, 5, and 24-hour treatments. Isolated preadipocyte cultures were plated into a 96-well plate and cultured overnight. The cells were treated with reconstituted powder formulation or saline-based tumescent solution for 1, 5 or 24 hours. Blank controls were generated for reconstituted powder formulation and saline with no cells. During the last hour, CTB was added to the cultures and blanks. After 1 hour, the plate was analyzed for cell viability. The results are shown in Table 4 and plotted as shown in FIG. 2. As shown in FIG. 2, there was minimal difference between treatments at 1 and 5 hours. At 24 hours, the Reconstituted Powder Formulation demonstrated an increase in viability while saline (control) showed almost complete toxicity. The media treatment (no txt, media) showed a basal level of metabolism that was considerably lower than the other samples, probably due to the cells reaching confluence and going dormant.

TABLE 4

| | | | CTB (RFU) | | |
|---|---|---|---|---|---|
| | | | ave | st dev | sem |
| Reconstituted | 1 | h | 10702.72 | 1419.135 | 819.3378 |
| Powder | 5 | h | 10159.51 | 120.1182 | 69.3503 |
| Formulation | 24 | h | 12414.85 | 885.9714 | 511.5159 |
| Saline | 1 | h | 11072.11 | 752.5388 | 434.4785 |
| | 5 | h | 9247.204 | 4850.895 | 2800.666 |
| | 24 | h | 205.134 | 40.6016 | 23.44135 |

| | | | | |
|---|---|---|---|---|
| media | no txt | 1281.979 | 139.9345 | 80.79124 |

### Experiment Example 2

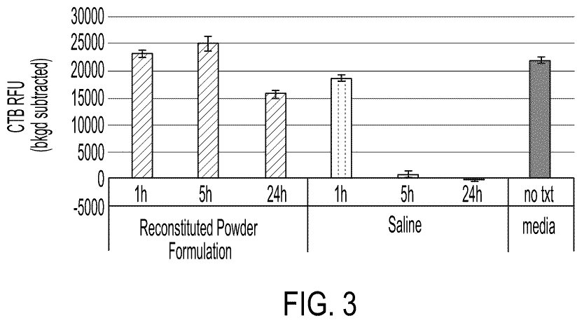

In vitro differentiated mature adipocyte viability after 1, 5, and 24-hour treatments. Isolated preadipocyte cultures were plated into a 96-well plate and cultured overnight. The cells were treated with Reconstituted Powder Formulation or saline for 1, 5 or 24 hours. The treatment was removed and cells were washed with PBS prior to addition of differentiation media. Cells were differentiated according to ZenBio™ protocol for 2 weeks. After 2 weeks CTB was added to the culture, and the plate was analyzed for cell viability after 2 hours. The results (after correcting for background fluorescence) are shown in Table 5, and plotted as shown in FIG. 3. As shown in FIG. 3, there was a significant increase in viability in adipocytes treated with reconstituted powder formulation compared to saline at all timepoints. Saline shows gross toxicity at all exposure times.

TABLE 5

| | Time | | CTB (RFU) | | |
|---|---|---|---|---|---|
| | (hr) | | ave | st dev | sem |
| Reconstituted | 1 | h | 23144.85 | 875.809 | 505.6485 |
| Powder | 5 | h | 24949.77 | 2146.518 | 1239.293 |
| Formulation | 24 | h | 15585.63 | 1227.513 | 708.7047 |
| Saline | 1 | h | 18455.88 | 906.6221 | 523.4385 |
| | 5 | h | 745.0037 | 1248.96 | 721.0876 |
| | 24 | h | −108.826 | 35.18735 | 20.31543 |

| | | | | |
|---|---|---|---|---|
| media | no txt | 21716.93 | 974.2556 | 562.4867 |

### Experimental Example 3

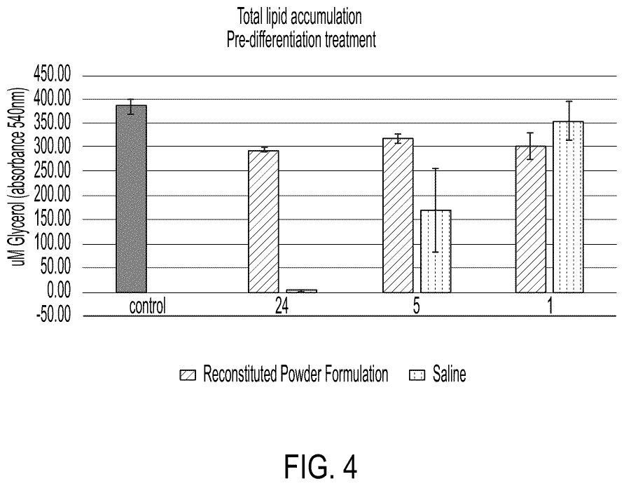

In vitro differentiated mature adipocyte lipid accumulation after 1, 5, and 24-hour treatments of preadipocytes. Isolated preadipocyte cultures were plated into a 96-well plate and cultured overnight. The cells were treated with reconstituted powder formulation or saline for 1, 5 and 24 hours. The cells were washed with PBS and treated with differentiation media for 7 days. The differentiation media was exchanged for maintenance media and cultured for an additional 7 days. After CTB assay, the cells were lysed and total triglyceride was measured according to ZenBio™ protocol. A standard curve was generated using stock concentrations of glycerol. Three replicates from each treatment time point were measured and mean concentrations of total glycerol are shown in Table 6, and plotted as shown in FIG. 4. Student's t-test was performed to compare differences from control (untreated cells) and treatment time points within each solution data set and time points were compared across treatment data sets. As shown in FIG. 4, reconstituted powder formulation showed no toxicity and lipid production after treatment of preadipocytes occurred with only modest reductions relative to the control for up to 24 hours. Saline treated cells, in contrast, showed inhibition of lipid accumulation at all time points, which may be due to the observed toxicity to preadipocytes and inhibition of adipocyte maturation. There are significant differences between the reconstituted powder formulation and saline solutions at all time points.

TABLE 6

| | | | Average | | |
|---|---|---|---|---|---|
| | | | μM | | |
| Compound | Time | | Glycerol | stdev | sem |
| Reconstituted | 24 | h | 385.01 | 50.22 | 16.74 |
| Powder | 5 | h | 294.40 | 7.72 | 4.46 |
| Formulation | 1 | h | 317.97 | 18.23 | 10.52 |

| | | | |
|---|---|---|---|
| no txt | 301.41 | 46.66 | 26.94 |

| | | | | | |
|---|---|---|---|---|---|
| Saline | 24 | h | | | |
| | 5 | h | 0.76 | 1.91 | 1.10 |
| | 1 | h | 168.92 | 148.40 | 85.68 |

| | | | |
|---|---|---|---|
| no txt | 353.64 | 68.80 | 39.72 |

### Experimental Example 4

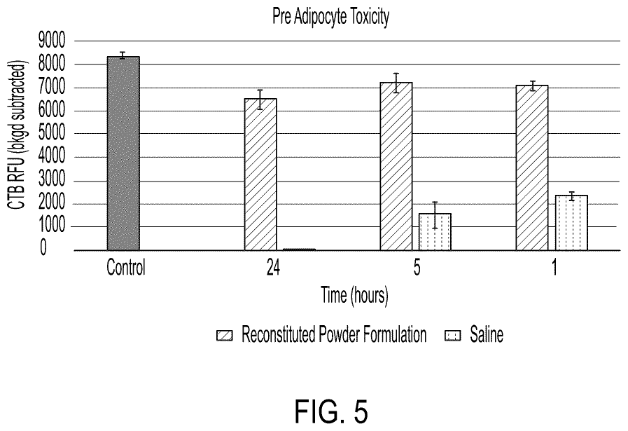

Preadipocyte viability after 1, 5, and 24-hour treatments using Cell Titer Blue. Isolated preadipocyte cultures were plated into a 96-well plate and cultured overnight. The cells were treated with reconstituted powder formulation or saline for 1, 5 and 24 hours. Blank controls were generated for reconstituted powder formulation and saline with no cells. After each treatment, cells were washed 2× with warm Hanks' Balanced Salt Solution (HBSS) and 1×CTB was added to the cultures and blanks. After 1 hour, the plate was analyzed for cell viability, and results corrected for background fluorescence. The results are shown in Table 7, and plotted as shown in FIG. 5. As shown in FIG. 5, there was substantial differences between treatments at 1 and 5 hours with reconstituted powder formulation compared to saline tumescent solution treated cells, with the saline tumescent treated cells losing substantial viability as soon as 1 hour after treatment. At 24 hours, cells treated with reconstituted powder formulation showed sustained viability while cells treated with saline showed almost complete toxicity.

TABLE 7

| | Time | CBT (RFU) | | |
|---|---|---|---|---|
| | (hr) | ave | stdev | sem |
| Control | Control | 8355.635 | 387.073 | 111.7383 |
| Reconstituted | 24 | 6471.618 | 734.6113 | 424.128 |
| Powder | 5 | 7179.775 | 721.7979 | 416.7302 |
| Formulation | 1 | 7063.284 | 348.3579 | 201.1246 |
| Saline | 24 | 215.8023 | 100.2185 | 57.8612 |
| | 5 | 6603.61 | 965.3875 | 557.3667 |
| | 1 | 7865.278 | 286.6966 | 165.5244 |

### Experimental Example 5

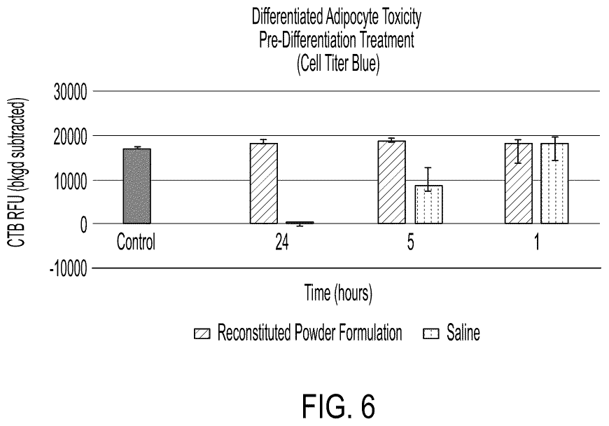

In vitro differentiated mature adipocyte viability after 1, 5, and 24-hour treatment prior to differentiation. Isolated preadipocyte cultures were plated into a 96-well plate and cultured overnight. The cells were treated with reconstituted powder formulation or saline for 1, 5 and 24 hours. The treatment solution was removed and cells were washed 1× with HBSS prior to addition of differentiation media. Cells were differentiated according to ZenBio™ protocol for 2 weeks. After 2 weeks CTB was added to the culture. After 1 hour of CTB addition, the plate was analyzed for cell viability. Results are shown in Table 8, and plotted as shown in FIG. 6. As shown in FIG. 6, there was a significant increase in viability in adipocytes treated with reconstituted powder formulation compared to adipocytes treated with saline at all time points. Saline treatment showed gross toxicity, especially at 5 and 24 hours.

TABLE 8

| | Time | CTB (RFU) | | |
|---|---|---|---|---|
| | (hr) | ave | stdev | sem |
| control | control | 17131.64 | 1379.499 | 398.227 |
| Reconstituted | 24 | 18306.12 | 1182.956 | 682.9798 |
| Powder | 5 | 18893.9 | 1120.019 | 646.6434 |
| Formulation | 1 | 18401.36 | 1111.487 | 641.7176 |
| Saline | 24 | 30.43133 | 142.768 | 82.42712 |
| | 5 | 8751.357 | 7159.378 | 4133.469 |
| | 1 | 18314.46 | 2735.304 | 1579.228 |

### Experimental Example 6

In Vitro Differentiated Mature Adipocyte Viability after 1, 5, and 24-Hour Treatment Prior to Differentiation.

Isolated preadipocyte cultures were plated into a 96-well plate and cultured overnight. The cells were treated with reconstituted powder formulation or saline for 1, 5 or 24 hours. The treatment solution was removed and cells were washed 2× with HBSS prior to addition of differentiation media. Cells were differentiated according to ZenBio™ protocol for 2 weeks. After 2 weeks, Live/Dead fluorescent stains were added to the culture for 20 minutes and imaged. The total number of cells was calculated and the percentage of cells containing Calcein AM (green fluorescence; live cells) or Ethidium dimer (red fluorescence; dead cells) were imaged.

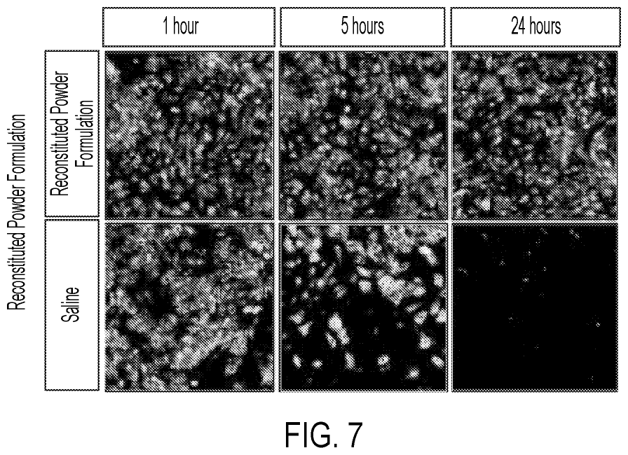

Representative images of 1, 5 and 24 h treatments are shown in FIG. 7 to compare culture conditions. In FIG. 7, live/dead stain was used to stain differentiated adipocytes for counting of mature adipocytes and fluorescent imaging of mature adipocytes. As shown in FIG. 7, viable adipocytes (green fluorescence) were observed when the reconstituted powder formulation was used for a period of 1 to 24 hours, whereas the viability of the adipocytes decreases over time, and was almost non-existent after 24 hours in saline solution (red fluorescence), which is shown by decreasing green fluorescence and increasing red fluorescence with increasing time of saline treatment.

### Experimental Example 7

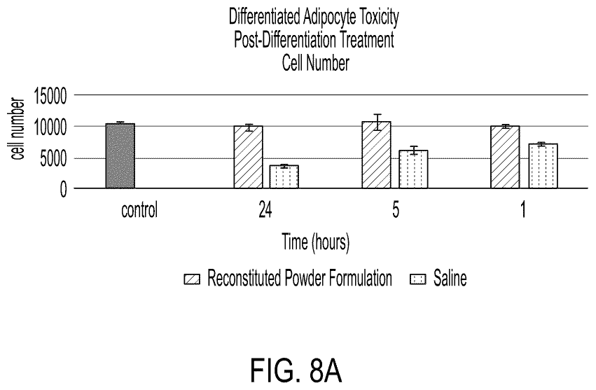

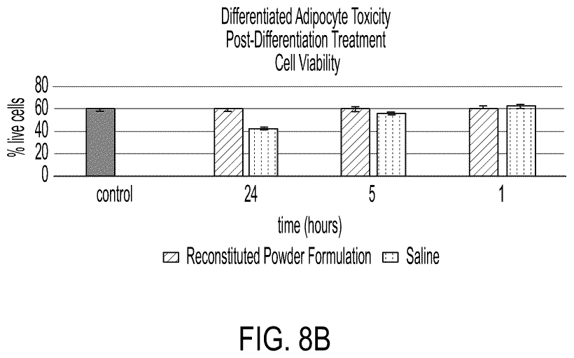

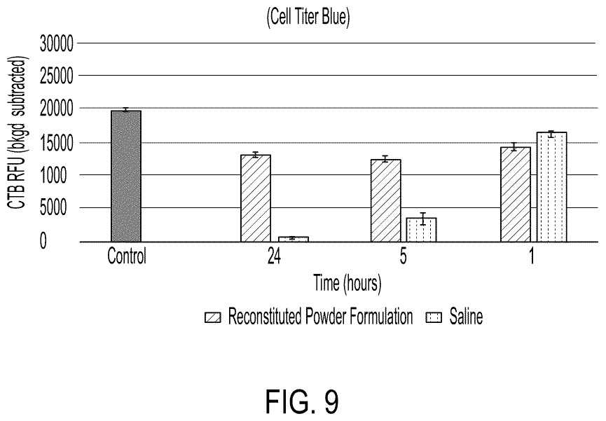

In vitro differentiated mature adipocyte viability after 1, 5, and 24-hour treatment post differentiation. Isolated preadipocyte cultures were plated into a 96-well plate and cultured overnight. Cells were differentiated according to ZenBio™ protocol for 2 weeks. The cells were then treated with reconstituted powder formulation or saline for 1, 5 and 24 hours. The treatment solution was removed and cells were washed twice with HBSS. CTB was added to the culture for 1 hour and the plate was analyzed for cell viability. The results are shown in Table 10, and plotted as shown in FIG. 9. Additionally, there was a significant increase in viability in adipocytes treated with reconstituted powder formulation compared to adipocytes treated with saline. This is consistent with the differences in cell number and cell viability (% live cells) shown in Tables 9A and 9B, and plotted in FIGS. 8A and 8B, respectively. As shown in FIGS. 8A and 8B, there was a significant increase in cell number for adipocytes treated with reconstituted powder formulation compared to adipocytes treated with saline at all timepoints with viability of remaining cells increasing for reconstituted powder formulation treated cells compared to saline treated cells at 5 and 24 hours. Overall, based on cell number and percentage of viable cells, viability increased for adipocytes treated with reconstituted powder formulation relative to adipocytes treated with saline at all timepoints.

TABLE 9A

| | Time | Number of Cells | | |
|---|---|---|---|---|
| | (hr) | ave | stdev | sem |
| control | control | 10472.25 | 633.05 | 182.759 |
| Reconstituted | 24 | 9859 | 900.952 | 520.16 |
| Powder | 5 | 10734.67 | 2034.62 | 1174.69 |
| Formulation | 1 | 9967.33 | 215.81 | 124.60 |
| Saline | 24 | 3518.33 | 300.16 | 173.30 |
| | 5 | 6245 | 672.79 | 388.449 |
| | 1 | 7083.67 | 330.03 | 190.547 |

TABLE 9B

| | Time | Percentage of live cells | | |
|---|---|---|---|---|
| | (hr) | ave | stdev | sem |
| control | control | 59.22 | 2.111 | 0.61 |
| Reconstituted | 24 | 59.02 | 0.64 | 0.37 |
| Powder | 5 | 60.14 | 3.27 | 1.89 |
| Formulation | 1 | 61.16 | 1.13 | 0.659 |
| Saline | 24 | 42.42 | 1.20 | 0.70 |
| | 5 | 56.543 | 1.90 | 1.10 |
| | 1 | 62.363 | 2.35 | 1.36 |

TABLE 10

| | Time | CBT (RFU) | | |
|---|---|---|---|---|
| | (hr) | ave | stdev | sem |
| control | Control | 19681.24 | 970.6973 | 280.2162 |
| Reconstituted | 24 | 12889.45 | 853.8564 | 492.9742 |
| Powder | 5 | 12217.3 | 816.3226 | 471.3041 |
| Formulation | 1 | 14188.38 | 1025.6 | 592.1305 |
| Saline | 24 | 381.695 | 72.04323 | 41.59418 |
| | 5 | 3225.581 | 1587.597 | 916.5996 |
| | 1 | 16230.64 | 821.6069 | 474.3549 |

### Experimental Example 8

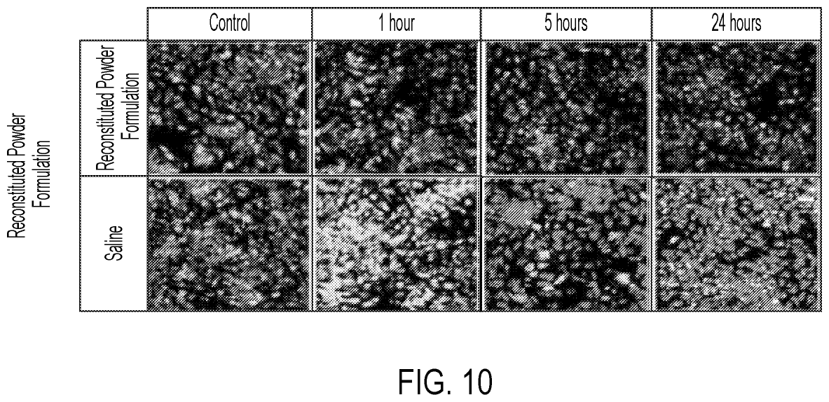

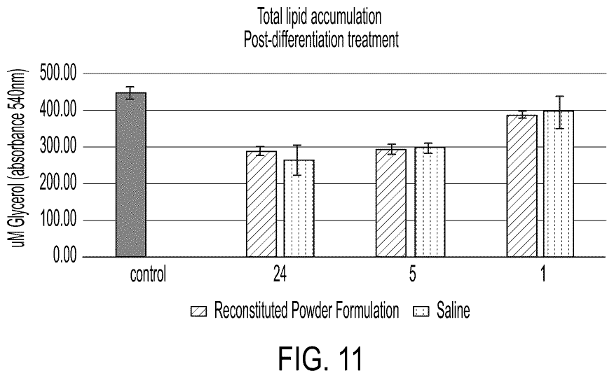

In vitro differentiated mature adipocyte viability after 1, 5, and 24-hour treatment post differentiation. Isolated preadipocyte cultures were plated into a 96-well plate and cultured overnight. Cells were differentiated according to ZenBio™ protocol for 2 weeks. The cells were then treated with reconstituted powder formulation or saline for 1, 5 and 24 hours. The treatment solution was removed and cells were washed twice with HBSS. Live/Dead fluorescent stains were added to the culture for 20 minutes and imaged. The total number of cells and the percentage of cells containing Calcein AM (green fluorescence; live cells) or Ethidium dimer (red fluorescence; dead cells) were calculated. Representative images of 24 h treatments are shown in FIG. 10 to compare culture conditions. As shown in FIG. 10, there was a significant increase in viability in adipocytes treated with reconstituted powder formulation (green fluorescence) compared to adipocytes treated with saline (red fluorescence). FIG. 10 shows a decreasing green fluorescence with increasing time of treatment with reconstituted powder formulation and an increasing red fluorescence with increasing time of treatment with a saline solution. Control represents cells containing only maintenance media. The total, post-differentiation lipid accumulation was measured, and the results are shown in Table 11 and FIG. 11. As shown in FIG. 11, the lipid accumulation under treatment conditions was similar to that observed for saline treatment at after 1 and 5 hours of treatment, and showed enhanced lipid accumulation after 24 hours of treatment.

TABLE 11

| | Time | Average μM Glycerol | | |
|---|---|---|---|---|
| | (hr) | ave | stdev | sem |
| control | control | 447.27 | 44.88 | 14.96 |
| Reconstituted | 24 | 288.35 | 21.14 | 12.20 |
| Powder | 5 | 293.45 | 24.57 | 14.19 |
| Formulation | 1 | 389.63 | 19.14 | 11.05 |
| Saline | 24 | | | |
| | 5 | 264.78 | 71.71 | 41.40 |
| | 1 | 297.27 | 21.98 | 12.69 |

As various changes could be made in the above formulations and methods without departing from the scope of the invention, it is intended that all matter contained in the above description shall be interpreted as illustrative and not in a limiting sense. Any numbers expressing quantities of ingredients, constituents, reaction conditions, and so forth used in the specification are to be interpreted as encompassing the exact numerical values identified herein, as well as being modified in all instances by the term “about.” Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C. § 112, paragraph 6, unless the term “means” is explicitly used.

1. A powder formulation, comprising: reduced glutathione; L-ascorbic acid; a sugar; L-arginine; a buffering agent; and a physically acceptable salt, wherein the powder formulation when reconstituted at a point of use in a saline or sterile water media forms a reconstituted formulation having a pH of about 7.

2-37. (canceled)
